(19) United States
(12) Patent Application Publication
(10) Pub. No.: US 2020/0289771 A1
(43) Pub. Date: Sep. 17, 2020

(54) MEDICAL DEVICE LOCATION TRACKING

(71) Applicant: Fisher & Paykel Healthcare Limited, Auckland (NZ)

(72) Inventors: David Martin Russell, Auckland (NZ); Gareth Alexander Clay, Auckland (NZ); Anton Kim Gulley, Auckland (NZ); Philip Ian Rowe, Auckland (NZ); Jin Kyu Lim, Auckland (NZ)

(21) Appl. No.: 16/765,635

(22) PCT Filed: Dec. 7, 2018
(86) PCT No.: PCT/IB2018/059740

§ 371 Date: May 20, 2020

Related U.S. Application Data
(60) Provisional application No. 62/596,592, filed on Dec. 8, 2017.

(51) Int. Cl. A61M 16/00; A61M 16/16; A61M 16/08; A61M 16/06; G16H 40/67; G16H 20/40; G05B 15/02; G01S 5/14; H04W 64/00

(52) U.S. Cl. CPC A61M 16/0003; G01S 5/0263; A61M 16/16; A61M 16/022; A61M 16/0051; A61M 16/0833; A61M 16/06; G16H 40/67; G16H 20/40; G05B 15/02; G01S 5/14; H04W 64/00; A61M 2205/8206; A61M 2205/3584; A61M 2205/52; A61M 2205/18; A61M 2205/581; A61M 16/0066

(57) ABSTRACT

Location tracking of medical devices or any consumer device. A location monitoring system can include a wireless communication system, such as a Global System for Mobile (GSM), a cellular system, a Wi-Fi system, or a global positioning system, programmed to transmit the device location under certain events. The system can also be configured to maintain the back up battery life by periodically turning a second global system for mobile communication on and off for a predetermined period of time. The system can also be configured to activate when the device is taken outside a predefined boundary, for example, a hospital.

### FIELD OF THE DISCLOSURE

The present disclosure relates to location tracking for medical devices. In particular, the present disclosure relates to location tracking in a respiratory flow therapy apparatus for delivering gas to patients.

### BACKGROUND

Flow therapy apparatuses are used in various environments such as hospital, medical facility, residential care, or home environments to deliver a flow of gas to users or patients. A flow therapy apparatus, or a respiratory device, may include a valve used to deliver supplementary oxygen with the flow of gas, and/or a humidification apparatus to deliver heated and humidified gases. A flow therapy apparatus may allow adjustment and control over characteristics of the gases flow, including flow rate, temperature, gases concentration, humidity, pressure, etc. Sensors, such as heated temperature sensing elements and/or thermistors, are used to measure these properties of the gases.

### SUMMARY

Medical devices are used for a number of different applications. Respiratory devices in particular are used to provide respiratory support to user's e.g. patients. Respiratory devices are meant to be used continuously by patients for extended periods of time. Respiratory devices can, for example be used to treat respiratory disorders such as Chronic Obstructive Pulmonary Disease (COPD) by delivering a flow of gas to users or patients. Respiratory devices can also be used to treat respiratory disorders such as Obstructive Sleep Apnea (OSA) by delivering pressurized gas to the airway of a user before and while they are asleep to splint the user or patient's airway. Respiratory devices can also include humidifiers that are used to humidify a flow of gases with water vapor prior to delivering to a patient. Humidifiers can be used with other respiratory devices that provide a gases flow.

The respiratory devices can be mounted on a mobile pole stand with wheels or are made mobile by other means (e.g. a handle or wearable) to allow patients to continue to use the device while moving about the hospital. Respiratory devices are also often used on a daily basis by patients. Home use devices are often moved with the user when the user travels for work, leisure or any other purpose. Because these devices are often not purchased by a user, but rather the user's insurance company, it is important to keep track of these relatively expensive medical devices. Unfortunately, users often misplace or lose these devices. Accordingly, the present disclosure provides for the ability to track these devices within a localized environment e.g. within the boundaries of a hospital or track the device outside of the boundaries of the hospital.

There are challenges in tracking devices when they are unplugged for cleaning and disinfection, as well as being stored or transported. Further, there are challenges in tracking respiratory devices when they are powered off for maintenance procedures. The present disclosure provides for the ability to track these devices while unplugged by means of an internal dedicated battery and communications device, such as a Global System for Mobile (GSM), any other cellular or wireless (e.g. Wi-Fi) system and/or global positioning system, which are programmed to transmit the devices location under certain events.

There are additional challenges in being able to monitor the usage of the respiratory devices. These tracking capabilities are only available when there is an available power source. This presents additional challenges in maintaining battery longevity for long term tracking. The systems described herein address the above issues by providing, for example, a method of maintaining a back up battery life by periodically turning a dedicated location monitoring system on and off for a predetermined period of time in accordance with specific events.

In one aspect, there is provided a respiratory device configured to provide respiratory flow therapy to a user, the respiratory device comprising;

a housing comprising a gas inlet and a gas outlet,

a wireless communication system disposed within the housing, the wireless communication system configured to receive a first signal from a first wireless transmitter,

a controller configured to communicate with the wireless communication system, the controller being configured to receive the first signal and determine a first measure of a first parameter associated with the first wireless transmitter,

wherein the controller is configured to use the first measure to estimate a first location, the first location providing an indication of the location of the respiratory device relative to the first wireless transmitter,

wherein the controller is configured to retrieve a first transmitter physical location indicative of the physical location of the first wireless transmitter, and

wherein the controller is configured to estimate a device physical location using the first location and the first transmitter physical location.

Optionally, the controller is configured to communicate with a memory, and wherein the controller is configured to retrieve the first transmitter physical location from the memory.

Optionally, the memory is an on-board memory unit located within the housing.

Optionally, the first parameter is associated with one or more of:

a) a received transmitter output as received by the wireless communication system, or

b) a time.

Optionally, the received transmitter output is a transmitter power output.

Optionally, the first measure is a measure of the received transmitter power output as received by the wireless communication system.

Optionally, the controller is configured to compare the first measure to a first threshold, and the controller is configured to estimate the first location based on the comparison.

Optionally, the first threshold is a value indicative of the received transmitter power output as received by the wireless communication system at a predefined distance.

Optionally, the controller is configured to apply a function to the first measure and the first threshold to estimate the first location.

Optionally, the first parameter is time, and the first measure is a transmission time indicative of the time the first signal was transmitted from the first wireless transmitter.

Optionally, the controller is configured to compare the transmission time to a received time, wherein the received time is indicative of a time the first signal was received by the wireless communication system, and the controller is configured to estimate the first location based on the comparison.

Optionally, the controller is configured to apply a function to the received time and the transmission time to estimate the first location.

Optionally, the wireless communication system is configured to receive a signal from each of a plurality of wireless transmitters.

Optionally, the device receives a unique signal from each wireless transmitter.

Optionally, the wireless communication system is configured to receive a second signal from a second wireless transmitter and a third signal from a third wireless transmitter.

Optionally, the controller is configured to receive the second signal and determine a second measure of a second parameter associated with the second wireless transmitter, and the controller is configured to receive the third signal and determine a third measure of a third parameter associated with the third wireless transmitter.

Optionally, each of the second parameter and the third parameter are associated with one or more of:

a) a received transmitter output as received by the wireless communication system, or

b) a time.

Optionally, the received transmitter output is a transmitter power output.

Optionally, the second measure is a measure of the received transmitter power output from the second wireless transmitter as received by the wireless communication system, and the third measure is a measure of the received transmitter power output from the third wireless transmitter as received by the wireless communication system.

Optionally, the controller is configured to compare the second measure to a second threshold, and the controller is configured to estimate the second location based on the comparison, and wherein the controller is configured to compare the third measure to a third threshold, and the controller is configured to estimate the third location based on the comparison.

Optionally, the second threshold is a value indicative of the received transmitter power output from the second transmitter as received by the wireless communication system at a second predefined distance, and the third threshold is a value indicative of the received transmitter power output from the third transmitter as received by the wireless communication system at a third predefined distance.

Optionally, the controller is configured to apply a function to the second measure and the second threshold to estimate the second location, and the controller is configured to apply a function to the third measure and the third threshold to estimate the third location.

Optionally, the second parameter is time, and the second measure is a second transmission time indicative of the time the second signal was transmitted from the second wireless transmitter, and wherein the third parameter is time, and the third measure is a third transmission time indicative of the time the third signal was transmitted from the third wireless transmitter.

Optionally, the controller is configured to compare the second transmission time to a second received time, wherein the second received time is indicative of a time the second signal was received by the wireless communication system, and the controller is configured to estimate the second location based on the comparison.

Optionally, the controller is configured to compare the third transmission time to a third received time, wherein the third received time is indicative of a time the third signal was received by the wireless communication system, and the controller is configured to estimate the third location based on the comparison.

Optionally, the controller is configured to apply a second function to the second received time and the second transmission time to estimate the second location.

Optionally, the controller is configured to apply a third function to the third received time and the third transmission time to estimate the third location.

Optionally, the controller is configured to use the first measure, the second measure and the third measure to estimate a relative location, the relative location providing an indication of the location of the device relative to the first wireless transmitter, the second wireless transmitter and the third wireless transmitter.

Optionally, the controller is configured to perform a trilateration calculation to estimate a relative location, the relative location providing an indication of the location of the device relative to the first wireless transmitter, the second wireless transmitter and the third wireless transmitter.

Optionally, the controller is configured to retrieve a first transmitter physical location indicative of a first physical location of the first wireless transmitter, a second transmitter physical location indicative of a second physical location of the second wireless transmitter, and a third transmitter physical location indicative of a third physical location of the third wireless transmitter.

Optionally, the controller is configured to estimate the device physical location within a localized environment using the relative location, the first transmitter physical location, the second location, the second transmitter physical location, the third location and the third transmitter physical location.

Optionally, the controller is configured to estimate the device physical location using trilateration.

Optionally, the controller is configured to communicate with a remote memory via the wireless communication system.

Optionally, the wireless communication system comprises one or more of:

a) a Wi-Fi system,

b) a cellular network system,

c) a GSM system, or

d) a BlueTooth® system.

Optionally, the respiratory device comprises a blower disposed within the housing, the blower configured to deliver air from the gas inlet to the gas outlet.

Optionally, the respiratory device comprises a humidification system.

Optionally, the controller is configured to disconnect the blower from power when the controller estimates the respiratory device's location to be outside an expected operating perimeter.

Optionally, the controller is configured to disconnect the humidification system from power when the controller estimates the respiratory device's location to be outside an expected operating perimeter.

Optionally, the controller is configured to transmit an alarm using the wireless communication system to a central monitoring system indicating the estimated location of the respiratory device.

Optionally, the respiratory therapy device is configured to output an audible alarm when the controller estimates the respiratory device's location to be outside an expected operating perimeter.

Optionally, the respiratory device is one or more of:

a) a continuous positive airway pressure device,

b) a Bi-Level positive airway pressure device,

c) a nasal high-flow device,

d) a non-invasive ventilation device, and

e) a ventilator.

In another aspect, there is provided a respiratory therapy system comprising:

a central monitoring system,

a respiratory device comprising;
a housing comprising a gas inlet and a gas outlet,
a wireless communication system disposed within the housing, the wireless communication system configured to receive a first signal from a first wireless transmitter, and configured to communicate first signal data to the central monitoring system,

wherein the central monitoring system comprises a controller, the controller being configured to receive the first signal data and determine from the first signal data a first measure of a first parameter associated with the first wireless transmitter,

wherein the controller is configured to use the first measure to estimate a first location, the first location providing an indication of the location of the respiratory device relative to the first wireless transmitter,

wherein the controller is configured to retrieve a first transmitter physical location indicative of a physical location of the first wireless transmitter, and

wherein the controller is configured to estimate a device physical location using the first location and the first transmitter physical location.

Optionally, the first signal data is transmitted from the respiratory device to the central monitoring system via the wireless communication system.

Optionally, the first signal data is transmitted from the respiratory device to the central monitoring system via a second wireless communication system.

Optionally, the controller is configured to communicate with a memory, and wherein the controller is configured to retrieve the first transmitter physical location from the memory.

Optionally, the first parameter is associated with one or more of:

a) a received transmitter output as received by the wireless communication system, or

b) a time.

Optionally, the received transmitter output is a transmitter power output.

Optionally, the first measure is a measure of the received transmitter power output as received by the wireless communication system.

Optionally, the controller is configured to compare the first measure to a first threshold, and the controller is configured to estimate the first location based on the comparison.

Optionally, the first threshold is a value indicative of the received transmitter power output as received by the wireless communication system at a predefined distance.

Optionally, the controller is configured to apply a function to the first measure and the first threshold to estimate the first location.

Optionally, the first parameter is time, and the first measure is a transmission time indicative of the time the first signal was transmitted from the first wireless transmitter.

Optionally, the controller is configured to compare the transmission time to a first received time, wherein the first received time is indicative of a time the first signal was received by the wireless communication system, and the controller is configured to estimate the first location based on the comparison.

Optionally, the controller is configured to apply a function to the first received time and the transmission time to estimate the first location.

Optionally, the wireless communication system is configured to receive a signal from each of a plurality of wireless transmitters.

Optionally, the device receives a unique signal from each wireless transmitter.

Optionally, the wireless communication system is configured to receive a second signal from a second wireless transmitter and a third signal from a third wireless transmitter.

Optionally, the controller is configured to receive second signal data and determine a second measure of a second parameter associated with the second wireless transmitter, and the controller is configured to receive third signal data and determine a third measure of a third parameter associated with the third wireless transmitter.

Optionally, the second signal data and the third signal data are transmitted from the respiratory device to the central monitoring system via the wireless communication system.

Optionally, the second signal data and the third signal data are transmitted from the respiratory device to the central monitoring system via a second wireless communication system.

Optionally, each of the second parameter and the third parameter are associated with one or more of:

a) a received transmitter output as received by the wireless communication system, or

b) a time.

Optionally, the received transmitter output is a transmitter power output.

Optionally, the second measure is a measure of the received transmitter power output from the second wireless transmitter as received by the wireless communication system, and the third measure is a measure of the received transmitter power output from the third wireless transmitter as received by the wireless communication system.

Optionally, the controller is configured to compare the second measure to a second threshold, and the controller is configured to estimate the second location based on the comparison, and wherein the controller is configured to compare the third measure to a third threshold, and the controller is configured to estimate the third location based on the comparison.

Optionally, the second threshold is a value indicative of the received transmitter power output from the second transmitter as received by the wireless communication system at a second predefined distance, and the third threshold is a value indicative of the received transmitter power output from the third transmitter as received by the wireless communication system at a third predefined distance.

Optionally, the controller is configured to apply a function to the second measure and the second threshold to estimate the second location, and the controller is configured to apply a function to the third measure and the third threshold to estimate the third location.

Optionally, the second parameter is time, and the second measure is a second transmission time indicative of the time the second signal was transmitted from the second wireless transmitter, and wherein the third parameter is time, and the third measure is a third transmission time indicative of the time the third signal was transmitted from the third wireless transmitter.

Optionally, the controller is configured to compare the second transmission time to a second received time, wherein the second received time is indicative of a time the second signal was received by the wireless communication system, and the controller is configured to estimate the second location based on the comparison.

Optionally, the controller is configured to compare the third transmission time to a third received time, wherein the third received time is indicative of a time the third signal was received by the wireless communication system, and the controller is configured to estimate the third location based on the comparison.

Optionally, the controller is configured to apply a second function to the second received time and the second transmission time to estimate the second location.

Optionally, the controller is configured to apply a third function to the third received time and the third transmission time to estimate the third location.

Optionally, the controller is configured to use the first measure, the second measure and the third measure to estimate a relative location, the relative location providing an indication of the location of the respiratory device relative to the first wireless transmitter, the second wireless transmitter and the third wireless transmitter.

Optionally, the controller calculates a trilateration calculation to estimate a relative location, the relative location providing an indication of the location of the respiratory device relative to the first wireless transmitter, the second wireless transmitter and the third wireless transmitter.

Optionally, the controller is configured to retrieve a first transmitter physical location indicative of the first physical location of the first wireless transmitter, a second transmitter physical location indicative of a second physical location of the second wireless transmitter, and a third transmitter physical location indicative of a third physical location of the third wireless transmitter.

Optionally, the controller is configured to estimate the device physical location within a localized environment using the relative location, the first transmitter physical location, the second location, the second transmitter physical location, the third location and the third transmitter physical location.

Optionally, the controller is configured to estimate the device physical location using trilateration.

Optionally, the wireless communication system comprises one or more of:

a) a Wi-Fi system,

b) a cellular network system,

c) a GSM system, or

d) a BlueTooth® system.

Optionally, the respiratory device comprises a blower disposed within the housing, the blower configured to deliver air from the gas inlet to the gas outlet.

Optionally, the respiratory device comprises a humidification system.

Optionally, the controller is configured to disconnect the blower from power when the controller estimates the respiratory device's location to be outside an expected operating perimeter.

Optionally, the controller is configured to disconnect the humidification system from power when the controller estimates the respiratory device's location to be outside an expected operating perimeter.

Optionally, the controller is configured to provide an alarm on the central monitoring system indicating the estimated location of the respiratory device.

Optionally, the central monitoring system is configured to output an audible alarm when the controller estimates the respiratory device's location to be outside an expected operating perimeter.

Optionally, the respiratory device is one or more of:

a) a continuous positive airway pressure device,

b) a Bi-Level positive airway pressure device,

c) a nasal high-flow device,

d) a non-invasive ventilation device, and

e) a ventilator.

In an aspect, there is provided a medical device comprising:

a first power source, and

a location monitoring system for providing the location of the medical device, the location monitoring system comprising:

a dedicated location monitoring system comprising a second wireless communication system and a second power source configured to power said dedicated location monitoring system.

Optionally, the location monitoring system further comprises a primary system comprising a first wireless communication system.

Optionally, the first power source comprises a mains power connection and/or a first internal battery.

Optionally, the primary system is powered by a mains power connection.

Optionally, the primary system is powered by the first internal battery when the mains power connection is disconnected.

Optionally, the second power source is a second internal battery.

Optionally, the second wireless communication system comprises a global system for mobile communications, a cellular system, a Wi-Fi system, a global positioning system, or any combination thereof.

Optionally, the first wireless communication system comprises a global system for mobile communications, a cellular system, a Wi-Fi system, a global positioning system, or any combination thereof.

Optionally, the second wireless communication system comprises a global system for mobile communications and determines the location of said medical device using cell tower triangulation.

Optionally, the dedicated location monitoring system remains inactive for a period of time.

Optionally, the period of time is a predetermined period.

Optionally, the medical device further comprising a third wireless communication system, the third wireless communication system comprises a Wi-Fi system and determines the location of said medical device.

Optionally, the medical device is a respiratory device.

In an aspect, there is provided a method of monitoring a location of a medical device, the method comprising:
detecting a charge of a first internal battery is below a certain threshold;
determining the location of said medical device using a second wireless communication system; and
reporting the location of said medical device to a monitoring system at a predetermined frequency.

Optionally, the second wireless communication system comprises a global system for mobile communications, a cellular system, a Wi-Fi system, or a global positioning system, or any combination thereof.

Optionally, the frequency of reporting is reduced based on the charge of a second internal battery.

Optionally, the medical device is a respiratory device.

Optionally, a primary system is powered by a mains power connection.

Optionally, a primary system is powered by the first internal battery.

Optionally the medical device described herein is configured to perform the method described earlier.

In an aspect, there is provided a method of monitoring a location of a medical device, the method comprising:
detecting a charge of a first internal battery is below a certain threshold;
determining the location of said medical device using a second wireless communication system; and
reporting the location of said medical device to a monitoring system at a predetermined frequency.

Optionally, the second wireless communication system comprises a global system for mobile communications, a cellular system, a Wi-Fi system, or a global positioning system, or any combination thereof.

Optionally, wherein the frequency of reporting is reduced based on the charge of a second internal battery.

Optionally, wherein the medical device is a respiratory device.

Optionally, wherein a primary system is powered by a mains power connection.

Optionally, wherein a primary system is powered by the first internal battery.

In another aspect, there is provided a method of monitoring a location of a medical device, the method comprising:
activating a dedicated location monitoring system after said dedicated location monitoring system remains inactive for a period of time;
determining a location of said medical device using the dedicated location monitoring system; and
reporting the location of said medical device to a central monitoring system.

Optionally, the method further comprising determining the location of said medical device at a predetermined frequency.

In another aspect, there is provided a method of monitoring a location of a medical device, the method comprising:
defining a geographical boundary;
determining a location of said medical device using a wireless communication system;
detecting the medical device has left the geographical boundary; and reporting the location of said medical device to a central monitoring system.

In another aspect, there is provided a method of monitoring a location of a medical device, the method comprising:
defining a geographical boundary;
determining a location of said medical device using a wireless communication system;
detecting the medical device has left the geographical boundary; and
disabling the medical device from further use.

The medical device as described earlier is configured to perform any one or more of the methods described in the various aspects herein.

Although the invention disclosed herein are directed to tracking respiratory devices in hospitals, the invention is not limited and may be applied to any medical device or other consumer product. Although the embodiments disclosed herein are directed to tracking respiratory devices in or outside of a hospital, the application is not limited and can be applied to any medical or consumer device in any setting.

### BRIEF DESCRIPTION OF THE DRAWINGS

Example embodiments that implement the various features of the disclosed systems and methods will now be described with reference to the drawings. The drawings and the associated descriptions are provided to illustrate embodiments and not to limit the scope of the disclosure.

FIG. 1 illustrates a block diagram of a primary and dedicated monitoring system.

FIG. 2 illustrates an embodiment of a process for detecting the location of a device.

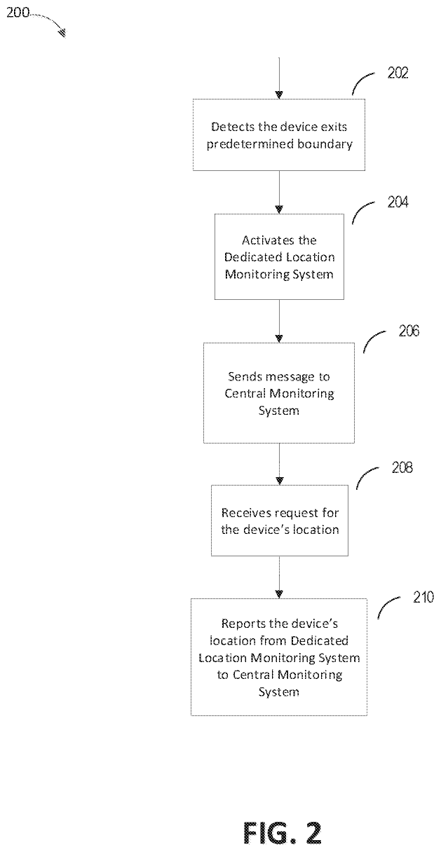

FIG. 3 illustrates an embodiment of a process for preserving battery life.

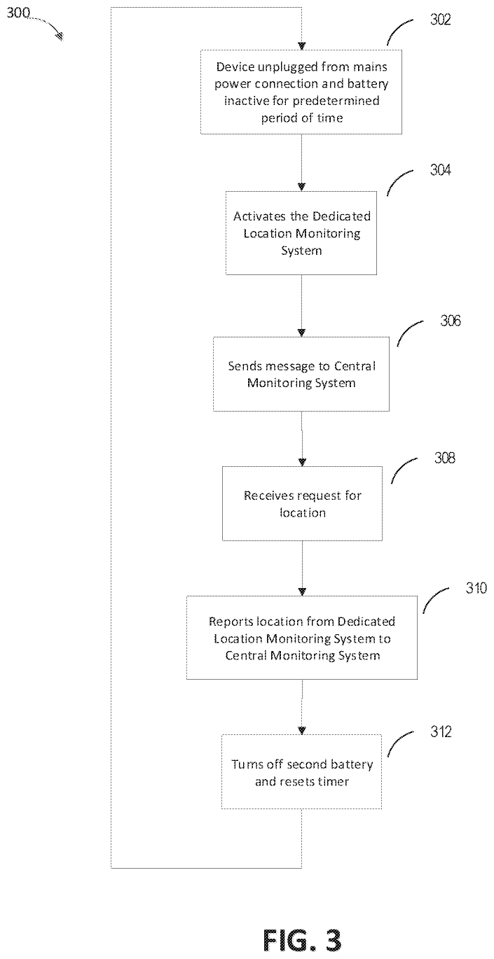

FIG. 4 illustrates a block diagram of a device comprising a wireless communication system and a local wireless network with a number of network nodes.

FIG. 5 illustrates a block diagram of a device comprising a wireless communication system, and a number of hardware transmitters.

FIG. 6 illustrates a block diagram of a device with a wireless communication system.

FIG. 7 is a schematic illustration of one form of a respiratory device.

FIG. 8 is a schematic illustration of one form of humidification system.

### DETAILED DESCRIPTION

The present disclosure relates to location tracking for medical devices. In particular, the present disclosure relates to location tracking in a respiratory device for delivering gas to a user. The respiratory device comprises a location tracking system to allow wireless location tracking of the respiratory device in a local environment (e.g. within a hospital or a private residence).

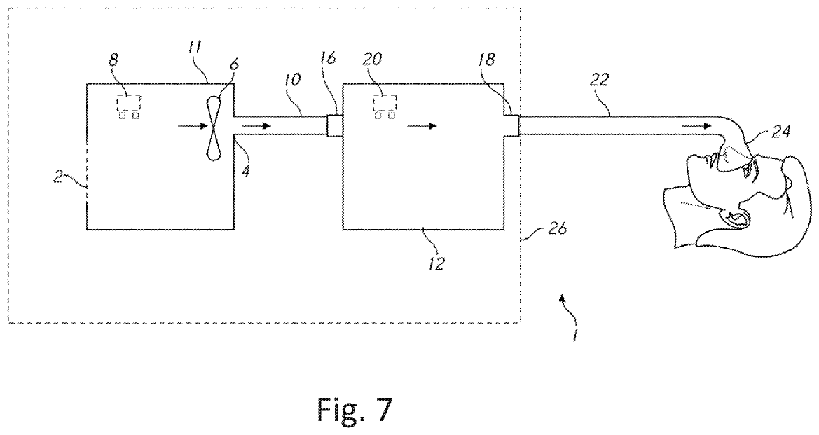

With reference to FIG. 7, a possible configuration for medical device is shown. The illustrated device is a respiratory device 1. The respiratory device 1 may be configured to provide respiratory therapy for the treatment of one or more respiratory conditions. The respiratory device 1 is configured to deliver or facilitate the delivery of respiratory gas to an airway of the user. For example, the respiratory device 1 can be a continuous positive airway pressure (CPAP) device configured to deliver a continuous stream of positive pressure breathing gas to a user to, for example, treat Obstructive Sleep Apnea (OSA). Alternatively, the respiratory device 1 can be configured to provide Bi-Level respiratory therapy to the user to treat OSA, for example. The Bi-Level respiratory therapy can include delivery of pressurized gas at a first pressure during inspiration of the user, and delivery of pressurized gas at a second pressure during exhalation of the user. The first pressure is generally higher than the second pressure. Alternatively, the respiratory device 1 can be configured to provide high-flow therapy to the user to treat ailments such as Chronic Obstructive Pulmonary Disorder (COPD). In such embodiments, the respiratory device 1 can deliver a flow of gas to the user's airways to assist in flushing out residual gas in the user's airway that has a relatively high CO₂ concentration. In one form, the respiratory device 1 can be a non-invasive ventilation device configured to deliver non-invasive ventilation to a user. In one form, the respiratory device 1 can be a ventilator. In the illustrated configuration, the respiratory device 1 may comprise a flow generator 11. The flow generator 11 may comprise a gas inlet 2 and a gas outlet 4. The flow generator 11 may comprise a blower 6. The blower 6 may comprise a motor. The motor may comprise a stator and a rotor. The rotor may comprise a shaft. An impeller may be linked to the shaft. In use, the impeller may rotate concurrently with the shaft to draw in gas from the gas inlet 2. The flow generator 11 may comprise a user interface 8 which may comprise one or more buttons, knobs, dials, switches, levers, touch screens, speakers, displays, and/or other input or output modules that a user might use to view data and/or to input commands into the flow generator 11 to control its operation and/or the operation of other components of the respiratory device 1. The flow generator 11 may pass gas through the gas outlet 4 to a first conduit 10. The first conduit 10 may pass the gas to a gas humidifier 12 that may be used to entrain moisture in the gas in order to provide a humidified gas stream. The gas humidifier 12 may comprise a humidifier inlet 16 and a humidifier outlet 18. The gas humidifier 12 may comprise fluid, such as water or another liquid or fluent solid suitable for use in gas humidification (elsewhere in this disclosure collectively referred to as water). The gas humidifier 12 may also comprise a heater that may be used to heat the water in the gas humidifier 12 to encourage water vaporization and/or entrainment in the gas flow and/or increase the temperature of gases passing through the gas humidifier 12. The heater may, for example, comprise a resistive heating element. The gas humidifier 12 may comprise a user interface 20 which may comprise one or more buttons, knobs, dials, switches, levers, touch screens, speakers, displays and/or other input or output modules that a user might use to view data and/or input commands into the gas humidifier 12 to control its operation and/or the operation of other aspects of the respiratory device 1. Various configurations for the gas humidifier 12 are described elsewhere in this disclosure and in the accompanying figures. For example, humidification system 800 may be the gas humidifier 12. Gas may then pass from the humidifier outlet 18 to a second conduit 22. The second conduit 22 may comprise a conduit heater.

The conduit heater may be used to add heat to gases passing through the second conduit 22. The heat may reduce or eliminate the likelihood of condensation of water vapour entrained in the gas stream along a wall of the second conduit 22. The conduit heating arrangement may comprise one or more resistive wires located in, on, around, or near a wall of the second conduit 22. Gas passing through the second conduit 22 may then enter a patient interface 24 that may pneumatically link the respiratory device 1 to an airway of a patient. The patient interface 24 may comprise a sealing or non-sealing interface. For example, the patient interface 24 may comprise a nasal mask, an oral mask, an oro-nasal mask, a full face mask, a nasal pillows mask, a nasal cannula, an endotracheal tube, a combination of any of the above, or some other gas conveying system or apparatus.

In the illustrated configuration, and as implied above, the respiratory device 1 may operate as follows. Gas may be drawn into the flow generator 11 through the gas inlet 2 due to the rotation of an impeller of the motor of the blower 6. The gas may then be propelled out of the gas outlet 4 and along the first conduit 10. The gas may enter the gas humidifier 12 through the humidifier inlet 16. Once in the gas humidifier 12, the gas may pass along a gas flow path to the outlet 18 of the humidifier. As the gas passes along the flow path, the gas entrains moisture when passing over or near fluid, such as water in the gas humidifier 12. Optionally, the water/fluid may be held within a water reservoir in the gas humidifier 12. The water may be heated by the heating arrangement, which may aid in the humidification and/or heating of the gas passing through the gas humidifier 12. The gas may leave the gas humidifier 12 through the humidifier outlet 18 and enter the second conduit 22. Gas may be passed from the second conduit 22 to the patient interface 24, where the gas may be taken into the patient's airways to aid in the treatment of respiratory disorders. To summarize, in use, gas may pass through a gas flow path extending from the gas inlet 2 of the flow generator 11 to the patient interface 24.

The illustrated configuration should not be taken to be limiting and many other configurations for the respiratory device 1 are possible. In some configurations, the flow generator 11 may, for example, comprise a source or container of compressed gas (e.g., air, oxygen, etc.). The flow generator 11 or the container may comprise a valve that may be adjusted to control the flow of gas leaving the container. In some configurations, the flow generator 11 may use such a source of compressed gas and/or another gas source in lieu of the blower 6. In some configurations, the blower 6 may be used in conjunction with another gas source. In some configurations, the blower 6 may comprise a motorized blower or may comprise a bellows arrangement or some other structure adapted to generate a gas flow. In some configurations, the flow generator 11 may draw in atmospheric gases through the gas inlet 2. In some configurations, the flow generator 11 may be adapted to both draw in atmospheric gases through the gas inlet 2 and accept other gases (e.g., oxygen, nitric oxide, carbon dioxide, etc.) through the same gas inlet 2 or a different gas inlet. In yet another form, gases (such as oxygen, nitric oxide, carbon dioxide, etc.) may be introduced downstream of the blower. For example, in Bi-level pressure therapy, supplemental oxygen can be introduced at the second conduit to be delivered with the heated and humidified gas.

In some configurations, the flow generator 11 and the gas humidifier 12 may be integrated or may share a housing 26. The housing 26 may be a rigid housing. For example, the housing 26 may be polycarbonate, or polypropylene. The housing 26 may be substantially inflexible. In some configurations, the first conduit 110 may not be present. In some such configurations, the flow generator 11 may, for example, directly communicate gases to the gas humidifier 12. In at least one configuration, the blower 6 may be removable from the respiratory device 1. In at least one configuration, the humidifier 12 may be removable from the respiratory device 1.

In some configurations, the respiratory device 1 may comprise a single user interface located on the flow generator 11, the gas humidifier 12, the first or second conduit 10, 22, the patient interface 24, or another component of the respiratory device 1. In some configurations, the operation of components of the respiratory device 1 may be actuated wirelessly using a user interface located on a remote computing device, which may be a tablet, a mobile phone, a personal digital assistant, or another device. In some configurations, the operation of the flow generator 11, of the gas humidifier 12, or of other components or aspects of the respiratory device 1 may be controlled by a controller. The controller may comprise a microprocessor. The controller may be located in or on the flow generator 11, the gas humidifier 12, or other components of the respiratory device 1 or on a remote computing device. In some configurations, multiple controllers may be used.

In some configurations, the respiratory device 1 may comprise one or more sensors for detecting various characteristics of gases in the respiratory device 1, including pressure, flow rate, temperature, absolute humidity, relative humidity, enthalpy, gas composition, oxygen concentration, and/or carbon dioxide concentration, one or more sensors for detecting various characteristics of the patient or of the health of the patient, including heart rate, EEG signal, EKG/ECG signal, blood oxygen concentration, blood CO2 concentration, and blood glucose, and/or one or more sensors for detecting various characteristics of gases or other objects outside the respiratory device 1, including ambient temperature and/or ambient humidity. One or more of the sensors may be used to aid in the control of components of the respiratory device 1, including the gas humidifier 12, through the use of a closed or open loop control system (e.g., through the use of the controller mentioned above). In some configurations, the respiratory device 1 may utilize a multi-limb system comprising inspiratory and expiratory gas passageways that may interface with one or more airways of the patient. In at least one configuration, the respiratory device 1 may comprise one or more wireless communication chipset(s). The controller may be configured to communicate with the wireless communication chipset(s) to derive information related to wireless networks, or to transmit information wirelessly. The wireless chipset(s) may comprise one or more of a BlueTooth® module, a Wi-Fi module and a Global System for Mobile communications (GSM) module.

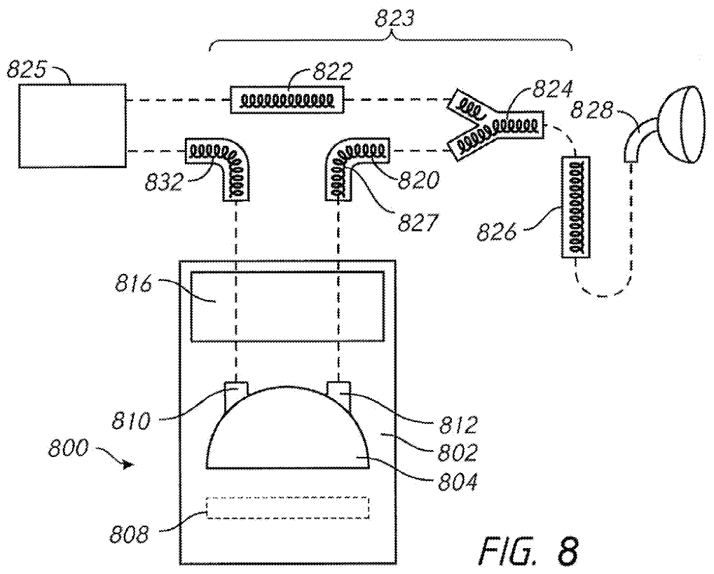

FIG. 8 schematically illustrates an example embodiment of a humidification system 800. The humidification system 800 can be used with the respiratory device 1, or another respiratory therapy device, breathing treatment system, positive pressure device, non-invasive ventilation device, and/or surgical procedures, including but not limited to laparoscopy. In some examples the humidifier 800 can be used with a ventilator or a wall gases source or an insufflator depending on the specific respiratory therapy being provided to a patient. In a further example the respiratory device 1 can comprise the humidification system 800. The humidification system 800 can be adapted to supply humidity or vapor to a supply of gases. The humidification system 800 can be particularly useful when used with a respiratory device that does not include its own humidification system. For example, in a case where the respiratory device does not include the gas humidifier 12, the humidification system 800 can provide humidity to gas flow.

An example embodiment of the humidification system 800 can include a heater base 802 and a humidification chamber 804. The heater base 802 can comprise a heater plate 808. The humidification chamber 804 can be configured to hold a volume of a liquid, such as water. The heater plate 808 can be configured to heat the volume of liquid held within the humidification chamber 804. The chamber 804 includes an inlet port 810 and an outlet port 812. The inlet port 810 receives gases into the chamber, the gases are humidified within the chamber and then outputted through the outlet port 812. The humidifier 802 comprises one or more sensors disposed on the humidifier to measure one or more parameters of the gases such as for example temperature, humidity, flow, gases concentration etc. In one example configuration the humidifier 802 comprises an inlet temperature disposed in the inlet port 810 and an outlet temperature disposed in the outlet portion 812. The humidifier 802 may also comprise a flow sensor to measure flow rate of the gases e.g. located in either of the inlet port 810 or outlet port 812. The humidifier 802 comprises a controller that controls power to the heater plate 808 based on the gas parameters determined by the various sensors on the humidifier, the heater plate being controlled to generate a desired or predefined amount of humidity. The humidifier 800 further comprises a screen 816 e.g. a touch screen to communicate information to users and receive inputs from users.

The humidification system 800 also can include a gases supply 825. In some configurations, the gases supply 825 can comprise a ventilator or any other suitable source of pressurized gases suitable for breathing or for use in medical procedures. The gases supply 825 can be separate from or combined with the heater base 802.

In some configurations, the humidification system 800 and/or the respiratory device 1 can include a breathing circuit or breathing circuit assembly 823. One or more of the components of the breathing circuit assembly 823 can be separable from, permanently coupled to or user-fitted to the chamber 804. The breathing circuit assembly 823 can include a second conduit 820 (i.e. inspiratory conduit). A chamber end of the second conduit 820 can be configured to connect to an outlet port 812 of the chamber 804. A patient end of the second conduit 820 can be configured to connect to the patient, for example, via an interface 828 (for example, nasal cannula, nasal pillows, full face mask, oral-nasal mask, oral interface, ET tube etc.). In some configurations, the second conduit 820 can be coupled directly to the interface 828. Any or all of the components of the breathing circuit assembly 823 can include a heating element, for example, a heating wire 827, to help maintain the gases at a desired temperature and to reduce the likelihood of significant condensation formation in the conduits. The second conduit 820 (i.e. inspiratory conduit) may include a sensor at the end of the conduit i.e. an end of hose sensor. The end of hose sensor is used to determine a property of the gases and control the heater wire 827 based on a feedback from the sensor.

In some configurations, for example, in configurations in which the gases supply 825 is separate from the heater base 802, the breathing circuit assembly 823 can include a first conduit 832. A gases supply end of the first conduit 832 can be configured to connect to an output of the gases supply 825 (e.g. the outlet of the blower 6). A chamber end of the first conduit 832 can be configured to connect to an inlet port 810 of the chamber 804. The first conduit 832 carries unhumidified gases or ambient air or a mixture thereof to the humidification chamber 804 for humidification. The first conduit 832 may be unheated or may optionally include a heater wire within the conduit to heat the gases being transported by the first conduit 832.

In some configurations, such as those used with a ventilator as the gases supply 825, the breathing circuit assembly 823 also can include an expiratory conduit 822. The humidification system used as part of an invasive ventilation set up will include the expiratory conduit 822. A patient end of the expiratory conduit 822 can be configured to connect to the interface 828 or connected to a gases manifold like the Y piece 824. A gases supply end of the expiratory conduit 822 can be configured to connect to a return of the gases supply 825.

In some embodiments, for example as shown in FIG. 8, the patient ends of the second conduit 820 and the expiratory conduit 822 can be connected to each other via a Y-piece 824. The Y-piece 824 can be connected to a patient interface conduit 826. In some configurations, the patient interface conduit 826 can include a catheter mount, for example but without limitation. The patient interface conduit 826 can be connected to the interface 828. In some embodiments, the Y-piece 824 couples to the interface 828 without an intervening patient interface conduit 826. The Y piece 824 and the patient interface conduit 826 may optionally include heater wires within them to maintain the gases passing through them at a desired temperature and to prevent condensation in these portions of the breathing circuit 823.

In some configurations, the heater base 802 can comprise a heater base display 116.

### Location Tracking

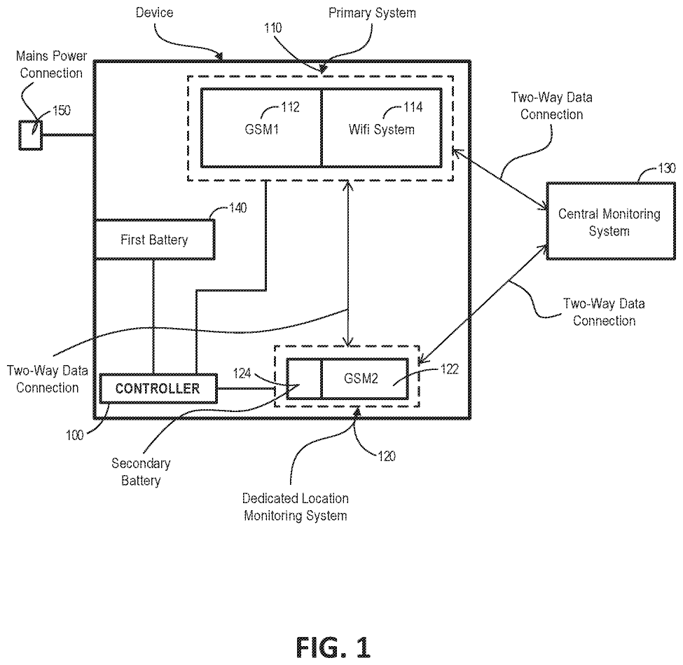

FIG. 1 illustrates a block diagram of an embodiment of a primary and dedicated monitoring system. The primary system 110 can include a first wireless communication system 112 and a second wireless communication system 114.

The primary and dedicated location monitoring systems for a device may be included within the housing of the device, attached to the outside of the device, mounted to a mobile pole stand, or attached by any other means. For example, the primary and dedicated location monitoring systems may be included within the housing of the respiratory device 1 previously described.

The system may include a controller 100 with one or more processors which are connected to a RAM or ROM or other non-volatile computer readable storage medium. The controller 100 may be configured to execute software, which includes instructions for determining and monitoring the location of a device. The controller 100 may also be configured to execute software, which includes instructions for controlling the operation of the device (e.g. respiratory device). The primary system 110 can be used for monitoring the location of the device. The controller 100 determines the location of the device using the primary system 110 and reports it to a centralized monitoring system 130. The centralized monitoring system 130 can comprise a remote server. The primary system 110 can use either a first wireless communication system 112 or a second wireless communication system 114 for location monitoring. The wireless communication systems can include a Global System for Mobile, any other cellular or Wi-Fi system and/or global positioning system, which are programmed to transmit the devices location under certain events. The wireless communication system 112 or 114 can comprise a Wi-Fi system, such that the location of the device can be monitored with its connection to the Wi-Fi network the device is located in. In at least one configuration, the first wireless communication system 112 is a cellular communication system. The cellular communication system can be a GSM communication system. In at least one configuration, the second wireless communication system 114 is a Wi-Fi system. For example, the router connected to the Wi-Fi system can be used to monitor the location of the device. The wireless communication system 112 or 114 can comprise a Global System for Mobile, such that the location of the device can be monitored through cell tower triangulation. For example, the first wireless communication device 112 can be used when the primary system is not plugged into the mains power source 150 or the Wi-Fi system 114 fails. Both systems in the primary system 110 require a power source. The power source can comprise a mains power source 150, an internal battery 140, the device itself, or any appropriate power source.

The location monitoring system can also include a dedicated location monitoring system 120 in addition to the primary system 110. The dedicated located monitoring system 120 can include a third wireless communication system 122 and a second internal battery 124. The wireless communication system can include a Global System for Mobile, any other cellular or Wi-Fi system and/or global positioning system, which are programmed to transmit the devices location under certain events. The controller 100 can use the dedicated location monitoring system 120 to monitor the location of the device and report the location to the centralized monitoring system 130. The controller 100 can use the third wireless communication system 122 in the dedicated location monitoring system 120 to monitor the location of the device if the primary system 110 fails for any reason.

### Battery Management

There is a need for improved battery management for location monitoring or tracking. Devices that are used long term and continuously, like respiratory devices, require long term battery management. Long term battery management increases the longevity of battery life. This allows for long term location tracking or the ability to track location when devices are lost.

When the device is connected to the mains power 150, the device's primary system 110 is powered and allows the location of the device to be monitored. For example, a wireless communication system that comprises a Wi-Fi system 114 can track the location of the device with its connection to the Wi-Fi network the device is located in. For example, the router connected to the device's Wi-Fi system 114 can be used to monitor the location of the device. Through the first wireless communication system 112, the controller 100 can use the first wireless communication system 112 for locating the device via cell tower triangulation. The controller 100 can still track the location of the device through the first wireless communication system 112, even when unplugged.

When the device is not connected to the mains power 150, the device 100 can be powered by the first internal battery 140. The first internal battery 140 can act as the primary system's back up power source.

The controller 100 can set the reporting frequency that the primary system 110 uses to report its location to a centralized monitoring system 130. The user may be a patient, hospital staff, or a provider of the device. The user can define this reporting frequency. The frequency of reporting can also be set at a default frequency. For example, the frequency the location can be reported at 10 minutes increments, every hour, every day, or another constant frequency or an irregular rate. The user can also request the location at any time, not at a predetermined rate. The controller 100 can also set the frequency the dedicated location monitoring system 120 reports the location to the centralized monitoring system 130. As another example, location reports can be sent more frequently when the device is first powered down and then can wait longer and longer time periods between reports to conserve battery. For example, the reports can start at 10 minute increments for the first 24 hours and then can switch to once a day and then once a month.

The controller 100 can detect the charge of the first internal battery 140 and send a message indicating the battery is low when it reaches a certain threshold. The message can prompt the user to request the location of the device. The message can report the location of the device. The threshold can be when the first internal battery 140 reaches a charge of 50%, 40%, 30%, 20%, 15%, 10% or any value therebetween. In some embodiments, the user can define the threshold value(s). The threshold value(s) can be set at default value(s).

When the primary system 110 is not connected to the mains power connection 150 and the first internal battery 140 dies, the controller 100 can use the dedicated monitoring system 120 to monitor the location of the device. The dedicated location monitoring system 120 can be powered by an independent second internal battery 124. The dedicated location monitoring system 120 also has an independent wireless communication system 122. The controller 100 can use the third wireless communication system 122 to monitor the location of the device if the primary system 110 fails for any reason. The controller 100 can report the location to the central monitoring system 130.

Both the primary and secondary battery 140, 124 can be rechargeable.

### Power Saving Mode

The device may be unplugged from a mains power supply 150 or the first internal battery 140 may fail or lose its charge, the dedicated location monitoring system 120 can be used to continue tracking the location of the device. It can be desirable to track the device for as long as possible using the secondary battery 124 in the back up system. To prolong the life of the secondary battery 124 and continue tracking of the device, the rate of reporting can be reduced to conserve the life of the second battery 124. For example, the rate of location reporting can be reduced by 50% of the rate which the primary system 110 reports the location of the device. The rate of reporting of the third wireless communication system 122 can be 50%, 40%, 30%, 20%, 15%, 10% or any value therebetween, of the rate of reporting of the primary system 110. The rate of reporting can be reduced to a constant rate of every day, every week, every month, or any other rate. The location reports can also include an indication of which system, primary or secondary, is being used to report the devices location. The location reports can also include the remaining battery life. The remaining battery life can be provided as a percentage of the total charge of the secondary battery 124, an estimate of the expected time in the future at which the charge of the secondary battery 124 will be too low to provide the location reports, another expression of the remaining battery life, or a combination of the possible indication methods. When location reports indicate a dying battery life, an alert can be sent to a monitoring service to indicate a danger that the device is lost. The monitoring service can use these alerts and reports to contact the user or operator and advise the user or operator of the location of the device.

The controller 100 can detect the charge of the second internal battery 124 of the dedicated location monitoring system 120. The controller 100 can detect that when the charge of the second internal battery 124 is below a certain threshold, the controller 100 can reduce the frequency to a predetermined rate. The threshold can be when the second internal battery 124 reaches a charge of 50%, 40%, 30%, 20%, 15%, 10% or any value therebetween.

The controller 100 can also detect the charge of the second internal battery 124 of the dedicated location monitoring system 120 and send a message to the central monitoring system 130 when it is below a certain threshold. The threshold can be when the second internal battery 124 reaches a charge of 50%, 40%, 30%, 20%, 15%, 10% or any value therebetween. The message can indicate the second internal battery 124 is low and prompt a user to request the location of the device. The message can indicate the second internal battery 124 is low and provide the location of the device. The message can indicate the second internal battery 124 is low and prompt a user to request to reduce the frequency of the rate of reporting the device's location to preserve battery power going forward. The message can prompt the user to reduce the frequency of the rate of reporting the device's location at a predetermined rate included in the message, or the message can prompt the user to reduce the frequency at a rate of the user's choice. For example, the message can prompt the user to reduce the frequency of the rate of reporting the device's location by a predetermined amount of 50%, 40%, 30%, 20%, 15%, 10% or any value therebetween. Alternately, upon being prompted, the user can elect to reduce the frequency of reporting by a user-selected amount on a spectrum of 0-100% or any value therebetween.

The controller 100 can detect when the charge of the first internal battery 140 and/or second internal battery 124 is below a certain threshold and emit an alarm. The threshold can be when the first internal battery 140 and/or the second internal battery 124 reaches a charge of 50%, 40%, 30%, 20%, 15%, 10% or any value therebetween. This alarm may be visual such as a color coded light, a flashing light, or a message on the screen of the device. This alarm may also be audio, such as a noise that is emitted at a certain threshold, certain thresholds, or periodically. The message can include contact information to inform a person that finds the device how to locate the device's owner or user.

The dedicated location monitoring system 120 can be defaulted to be powered off as the system uses the primary system 110 to track location. After a predetermined period of time, the controller 100 automatically activates the dedicated location monitoring system 120, detects the location of the device, and reports the location to the central monitoring system 130. The predetermined period of time can be 1 day, 1 week, 1 month, 3 months, 6 months, 1 year, or any value therebetween. The predetermined period of time can also be based on percentage of battery life remaining. The controller 100 can deactivate the dedicated location monitoring system 120 and reset the timer. The process 300 can repeat itself, such that the dedicated location monitoring system 120 is deactivated for the predetermined period of time. This cycle in which the controller 100 periodically turns on and off the dedicated location monitoring system 120 for a predetermined period of time to report the location to the central monitoring system 130 prolongs the internal second battery 124 lifespan.

### Other Uses:

The controller 100 can report the location of the device from the primary system 110 or the dedicated location monitoring system 120 when requested by the central monitoring system 130. The controller 100 can respond to intervention over the central monitoring system 130 without user intervention on the device side.

The controller 100 can detect when the device exits a defined geographical boundary. For example, if the controller 100 detects the device has been taken outside the boundaries of the hospital, the controller 100 can send a message to a central monitoring service that the device has been taken outside the hospital. The controller 100 can send a message to prompt a user to request a location over the central monitoring system 130. The controller 100 can also determine the location of the device using the first wireless communication system 112, the second wireless communication system 114, or the third wireless communication system 122. The controller 100 can send a message to the central monitoring system 130 and report the device's location.

The controller 100 can remotely lock the device so it cannot be used when taken outside the defined geographical boundary, or can provide a prompt to the user to remotely lock the device.

Location tracking also allows monitoring of usage of the respiratory device. The first wireless communication 112 can comprise a Wi-Fi system that can connect the device to a local Wi-Fi network. The primary system 110 can also be used to collect and report usage data of the device, respond to user intervention commands to monitor and report location, give over the air updates without user intervention on the device side, and give over the air updates in response to intervention over the central monitoring system 130.

### Location Tracking Methods

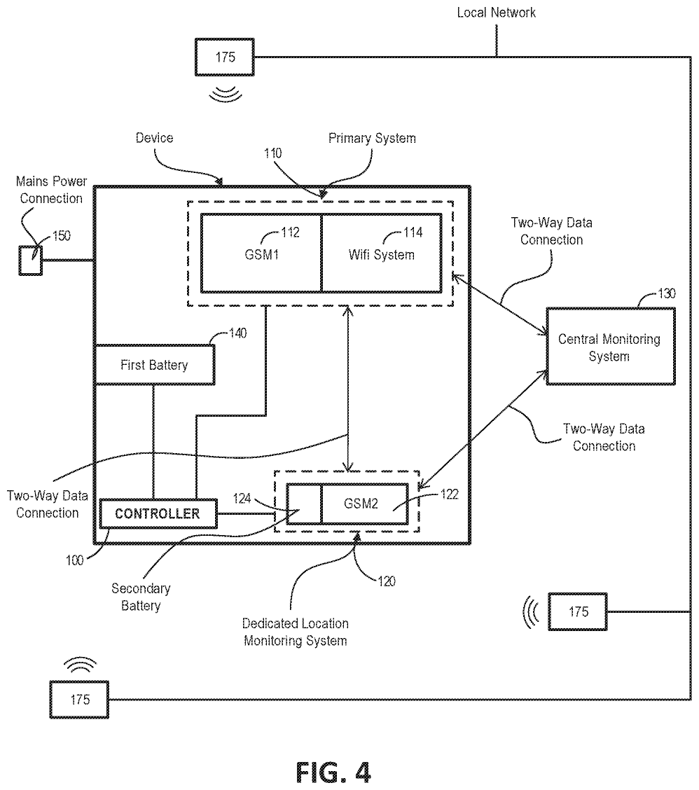

As previously disclosed, the device's primary system 110 allows the location of the respiratory device to be monitored. This can be particularly useful for tracking the location of a respiratory device. Referring to FIG. 4, one or more of the first wireless communication system 112 or second wireless communication system 114 can track the location of the respiratory device by receiving information from, or communicating with a local wireless network (e.g. Wi-Fi network, GSM network) the respiratory device is located within. The local wireless network can comprise one or more local nodes or wireless access points. In one form, the one or more local nodes or wireless access points can be in the form of or comprise one or more wireless transmitters. In at least one configuration, the local wireless network can be the hospital's local Wi-Fi network. In at least another configuration, the local wireless network can be a patient's home Wi-Fi network. The local wireless network can comprise one or more wireless access points (WAPs) 175. The WAPs 175 can be configured to provide access to the local wireless network. Preferably, the local wireless network can comprise a plurality of WAPs 175. For example, the hospital's local wireless network can comprise a plurality of WAPs 175, being Wi-Fi routers and/or modems.

The controller 100 can use the first wireless communication system 122 and/or the second wireless communication system 114 for locating the respiratory device. More specifically, the controller 100 can use the GSM system 112 and/or the Wi-Fi system 114 for locating the respiratory device. For example, if the respiratory device is located within a local Wi-Fi network, the controller 100 can use the second wireless communication system 114 to monitor the location of the respiratory device. The Wi-Fi system 114 can receive information and/or communicate with one or more of the WAPs 175. The Wi-Fi system 114 does not necessarily need to be granted access to the local Wi-Fi network by the WAPs 175. The Wi-Fi system 114 can provide to the controller 100 data related to each WAP 175 from which it receives information and/or communicates with. For example, the controller 100 can receive WAP 175 identification information from the Wi-Fi system 114. The WAP 175 identification information can include each WAP's 175 Service Set Identifier (SSID) and/or media access control (MAC) address. The Wi-Fi system 114 is therefore configured to provide WAP identification information related to WAPs 175 detectable by the Wi-Fi system 114 to the controller 100. The Wi-Fi system 114 can provide a measure of the received signal strength (from the WAP 175) to the controller 100. In at least one form, the Wi-Fi system 114 can provide an arbitrary strength unit (ASU) to the controller indicative of the strength of the signal received from each WAP 175. The ASU can be mapped to a received signal strength indication (RSSI) value, or each WAP 175 can provide an RSSI value indicative of the signal strength of the WAP 175 at a known and/or predefined distance. In at least one form, the Wi-Fi system 114 can provide an indication of the power of the received signal (e.g. in Watts) to the controller 100. In either case, the controller 100 is configured to process the WAP identification information and signal strength information and estimate a relative location of the respiratory device with respect to the WAP 175 from which the signal was received.

The respiratory device can use the RSSI method of localization. The local Wi-Fi network can comprise a first WAP 175a, a second WAP 175b and a third WAP 175c. The Wi-Fi system 114 provide a measure of the strength of a first signal received the first WAP 175a, a second signal received from the second WAP 175b and a third signal received from the third WAP 175c to the controller 100.

The controller can comprise and/or access a memory, such as a RAM or ROM or other non-volatile computer readable storage medium. The memory can include data indicative of the physical location of each of a number of WAPs 175. For example, the memory can include and/or store geo-location data (for example, GPS coordinates) related to the one or more WAPs 175 of the local Wi-Fi network. As such, the memory can include or store pre-determined GPS coordinates that indicate the position of the first WAP 175a, the second WAP 175b and the third WAP 175c. In another form, the memory can include and/or store location data comprising a map of a structure, correlating each particular WAP 175 to a room of the structure within which it is located. As a result, the controller can access and/or retrieve data indicative of the physical location, or relative location within a structure of each of the WAPs from the memory.

In one form, the controller 100 can apply a model to the measures of signal strength to estimate the distance between the respiratory device and the WAPs 175. The model can be a signal propagation model. The controller 100 can use one or more outputs of the model, and the data retrieved from the memory to estimate the physical location of the respiratory device. In other words, the controller 100 can employ trilateration to estimate the physical location of the respiratory device. The controller 100 can provide the determined physical location of the respiratory device to the central monitoring system 130 by transmitting the determined location over a wireless communication network, for example the GSM network, cellular system and/or a Wi-Fi system. For example, the controller 100 can transmit the physical location of the respiratory device to the central monitoring system 130 via the first wireless communication system 112. Alternately, the respiratory device can transmit the physical location of the respiratory device to the central monitoring system 130 via the second wireless communication system 114.

In an alternate configuration, the respiratory device can provide the WAP 175 identification information and/or the measure of signal strength from each WAP 175 (e.g. the ASU and/or the RSSI value) to the central monitoring system 130. The central monitoring system 130 can comprise and/or access a memory such as a RAM or ROM or other non-volatile computer readable storage medium. The memory can include data indicative of the location of each of a number of pre-programmed WAPs 175. For example, the memory can include and/or store geo-location data (for example, GPS coordinates) related to the one or more WAPs 175 of the local Wi-Fi network. For example, the memory can include or store pre-determined GPS coordinates that indicate the position of the first WAP 175a, the second WAP 175b and the third WAP 175c. As a result, the central monitoring system 130 can comprise the controller 100 which can access and/or retrieve data indicative of the location of each of the WAPs from the memory. In such a configuration, the central monitoring system 130 can use the information provided by the respiratory device to determine the respiratory device's location.

Use of the second wireless communication system 114 to estimate the physical location of the respiratory device can be advantageous in urban areas where other location tracking systems have reduced accuracy. Large artificial structures such as buildings can reduce the effectiveness of some location tracking methods such as GPS trilateration. As such, local wireless networks such as local Wi-Fi networks can provide improved location tracking performance. Use of a local Wi-Fi network to locate the respiratory device can improve the speed and/or accuracy at which the respiratory device is located.

In one form, the controller 100 can use the first wireless communication system 122 for locating the respiratory device. More specifically, the controller 100 can use the GSM system 112 for locating the respiratory device. The GSM system 112 can receive information and/or communicate with one or more nodes of a local GSM network. In one form, the nodes of the local GSM network can be in the form of WAPs 175. The GSM system 112 can provide to the controller 100 data related to each WAP 175 from which it receives information and/or communicates with. The GSM system 112 is therefore configured to provide WAP identification information related to WAPs 175 detectable by the GSM system 112 to the controller 100. The GSM system 112 can also provide a measure of the received signal strength (from the WAP 175) to the controller 100. For example, the GSM system 112 can provide an arbitrary strength unit (ASU) to the controller indicative of the strength of the signal received from each WAP 175. The ASU can be mapped to a received signal strength indication (RSSI) value, or each WAP 175 can provide an RSSI value indicative of the signal strength of the WAP 175 at a known and/or predefined distance. In at least one form, the GSM system 112 can provide an indication of the power of the received signal (e.g. in Watts) to the controller 100. In either case, the controller 100 is configured to process the WAP identification information and signal strength information and estimate a physical location of the respiratory device.

The respiratory device can use the RSSI method of localization. The local network (e.g. the GSM network) can comprise a first WAP 175a, a second WAP 175b and a third WAPc. The GSM system 112 can measure the strength of a first signal received the first WAP 175a, a second signal received from the second WAP 175b and a third signal received from the third WAP 175c. In other words, the GSM system 112 can measure the strength of a signal received from one or more WAPs. The GSM system 112 can provide the measure of the one or more signals to the controller 100.

The controller 100 can comprise and/or access a memory, such as a RAM or ROM or other non-volatile computer readable storage medium. The memory can include data indicative of the location of each of a number of WAPs. For example, the memory can include and/or store geo-location data (for example, GPS coordinates, latitude and longitude) related to the one or more WAPs 175 of the local GSM network. For example, the memory can include or store pre-determined GPS coordinates that indicate the position of the first WAP 175a, the second WAP 175b and the third WAP 175c. As a result, the controller can access and/or retrieve data indicative of the location of each of the WAPs from the memory.

The controller 100 can apply a model to the measures of signal strength to determine the distance between the respiratory device and the WAPs 175. The model can be a signal propagation model. The controller 100 can use one or more outputs of the model, and the data retrieved from the memory to estimate the physical location of the respiratory device. In other words, the controller 100 can employ trilateration to estimate the physical location of the respiratory device. The controller 100 can provide the estimated physical location of the respiratory device to the central monitoring system 130 by transmitting the estimated location over a wireless communication network, for example the GSM network, cellular system and/or a Wi-Fi system. For example, the controller 100 can transmit the physical location of the respiratory device to the central monitoring system 130 via the first wireless communication system 112. Alternately, the respiratory device can transmit the physical location of the respiratory device to the central monitoring system 130 via the second wireless communication system 114.

In an alternate configuration, the respiratory device can provide the WAP 175 identification information and/or the measure of signal strength from each WAP 175 (e.g. the ASU and/or the RSSI value) to the central monitoring system 130. The central monitoring system 130 can comprise the controller 100 which can access a memory such as a RAM or ROM or other non-volatile computer readable storage medium. The memory can include data indicative of the location of each of a number of pre-programmed WAPs 175. For example, the memory can include and/or store geo-location data (for example, GPS coordinates) related to the one or more WAPs 175 of the local GSM network. For example, the memory can include or store pre-determined GPS coordinates (latitude and longitude) that indicate the position of the first WAP 175a, the second WAP 175b and the third WAP 175c. As a result, the central monitoring system 130 can access and/or retrieve data indicative of the location of each of the WAPs from the memory. In such a configuration, the central monitoring system 130 can use the information provided by the respiratory device to estimate the respiratory device's location. This can be done as previously described.

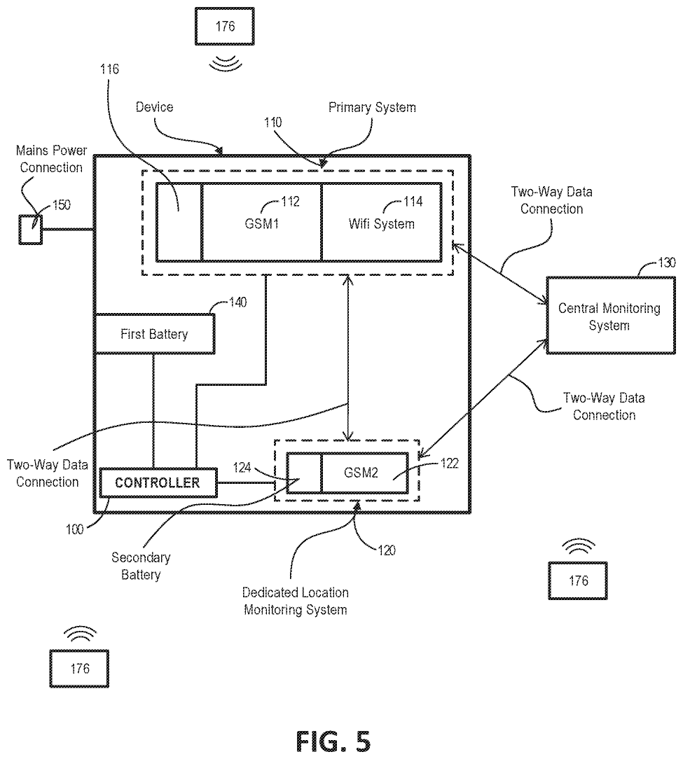

In an alternate configuration, as shown in FIG. 5, the primary system 110, comprises a fourth wireless communication system 116. The fourth wireless communication system 116 can comprise an ultra high frequency (UHF) transceiver system. In one form, the fourth wireless communication system 116 can be a BlueTooth® system. In one form, the fourth wireless communication system 116 is configured to communicate with or receive information from one or more nodes of a local network. In one form, the one or more nodes of the local network can be in the form of one or more wireless transmitters. In one form, the one or more nodes of the local network can be in the form of one or more hardware transmitters 176. The fourth wireless communication system 116 is configured to communicate with or receive information from the one or more hardware transmitters 176. The one or more hardware transmitters 176 can be BlueTooth® low energy devices. For example, the one or more hardware transmitters 176 can be BlueTooth® low energy beacons. The hardware transmitters 176 can be configured to broadcast an identifier. For example, each hardware transmitter 176 can be configured to transmit a universally unique identifier and/or information related to the respective hardware transmitter 176.

The respiratory device can be configured to estimate its physical location using the fourth wireless communication system 116 and a signal from the hardware transmitter/s 176. In one form, the fourth wireless communication system 116 can detect the transmitted signal from one of the hardware transmitters 176. The detection of the signal can be used as an indication that the respiratory device is located within a specific radius of the hardware transmitter 176, whose position is known. For example, in a hospital environment, one or more wards of the hospital can include their own hardware transmitter 176. The respiratory device detecting that it is in the proximity of a hardware transmitter 176 that is known to be in a particular ward, can then indicate to the central monitoring system 130 that the respiratory device is in said ward. Alternatively, the respiratory device can transmit the detected signal to the central monitoring system 130 which can contain a database of the signals transmitted from the hardware transmitter/s 176. Knowing the signal detected by the respiratory device, and the physical location of the hardware transmitter/s 176, the central monitoring system 130 can estimate the physical location of the respiratory device.

In at least another configuration, each hardware transmitter 176 can transmit an RSSI value indicative of the signal strength of the hardware transmitter 176 at a known distance. The fourth wireless communication system 116 can measure the strength of a signal received from one of the hardware transmitters 176. The fourth wireless communication system 116 can provide the measure of the signal strength and the RSSI value to the controller 100. The controller 100 can estimate the distance between the respiratory device and the hardware transmitter 176 using the measured signal strength and the RSSI value. Where multiple hardware transmitters 176 are detected by the fourth wireless communication system 116, trilateration can be used to estimate the respiratory device's physical location. For example, the respiratory device can be configured to estimate its physical location within a hospital. The controller 100 can comprise and/or access a memory. The memory can include pre-programmed data indicative of the location of each of the hardware transmitters 176. The controller 100 can compare the information provided by the one or more of the wireless communication systems (e.g. the fourth wireless communication system 116) with the pre-programmed data indicative of each hardware transmitter's 176 location to estimate the location of the respiratory device. For example, the memory can include and/or store geo-location data (for example, GPS coordinates, latitude and longitude) related to the one or more of the hardware transmitters 176.

In an alternate configuration, the respiratory device can communicate with the central monitoring system 130, which stores the pre-programmed data indicative of the location of each of the hardware transmitters 176. The respiratory device can provide the central monitoring system 130 with the detected hardware transmitter 176, and the detected signal strength and/or RSSI values of nearby hardware transmitters 176, and the central monitoring system 130 can estimate the location of the respiratory device. In such a form, the central monitoring system 130 can comprise the controller 100. In one form, the hospital can include one or more of the hardware transmitters 176 distributed throughout one or more rooms to allow location tracking as previously described.

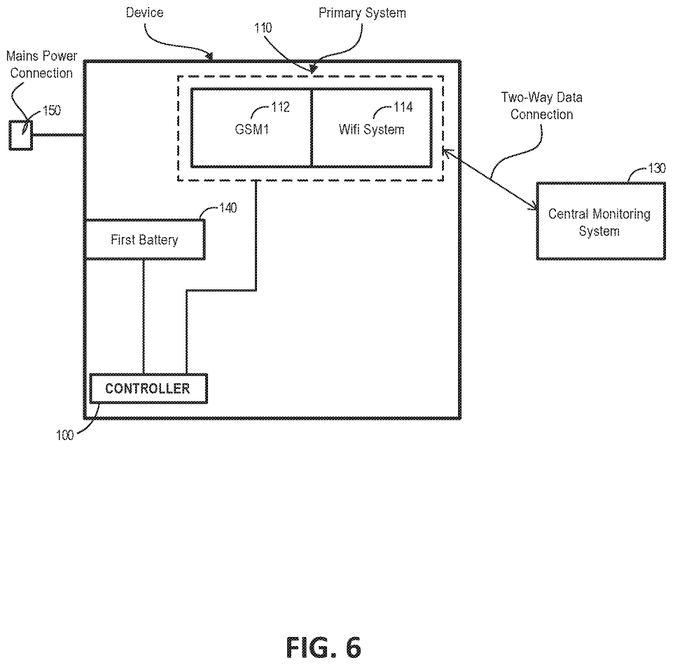

In at least one embodiment, the dedicated location monitoring system 120 is optional. For example, in at least one embodiment, the respiratory device and/or system does not include the dedicated location monitoring system 120. Such a system can include the primary system 110. The primary system 110 can include one or more of the first wireless communication system 112, the second wireless communication system 114 and the fourth wireless communication system 116. FIG. 6 shows an illustration of such a system, comprising the first, second and fourth wireless communication systems 112, 114, 116.

In at least one embodiment, a time differential of arrival (TDOA) method can be used to estimate the location of the respiratory device. In one form, the TDOA method can be employed by the controller 100 using one or more of the first wireless communication system 112, the second wireless communication system 114, the third wireless communication system 122 and the fourth wireless communication system 116. A signal sent from a node of a local network, for example a WAP 175 as previously described, a cell tower or a hardware transmitter 176 as previously described can include data comprising an identifier and a time. The identifier can uniquely identify the network node. The time can be the current time (e.g. the Coordinated Universal Time (UTC) time at which the signal was transmitted from the node). Alternatively, the time can be an internal system time to which all devices are synchronized.

The controller 100 can include a time keeping module. Alternatively, the controller 100 can be in communication with a time keeping module that is configured to provide the time to the controller 100. For example, the respiratory device can include a time keeping module that is configured to provide the time to the controller 100. One or more of the first, second, third and fourth wireless communication systems 112, 114, 122, 116 can receive the signal transmitted by the network node, and communicate the data of the signal to the controller 100. The controller 100 can compare the time of arrival of the signal from the network node (as recorded on the respiratory device) to the time the signal was transmitted (which is contained in the signal). The controller 100 can then estimate the location of the respiratory device with reference to the network node. In the case where a signal is transmitted and received from a single network node, the controller 100 can estimate a radius at or within which the respiratory device may be located. This can be done based on the difference between the time of arrival of the signal from the network node (as recorded on the respiratory device) to the time the signal was transmitted (which is contained in the signal). The controller 100 can comprise and/or access a memory, such as a RAM or ROM or other non-volatile computer readable storage medium. The memory can include data indicative of the location of each of a number of network nodes (for example, WAPs 175 and/or hardware modules 176). For example, the memory can include and/or store geo-location data (for example, GPS coordinates) related to the one or more local network nodes. The controller 100 can access the data from the memory indicative of the location of the network node from which the signal was received. The controller 100 can estimate the physical location of the respiratory device as at or within a calculated distance from the physical location of the network node. The controller 100 can transmit the estimated physical location of the respiratory device to the central monitoring system 130. The controller 100 can utilize one or more of the first, second, third and/or fourth wireless communication systems 112, 114, 122, 116 to transmit the location to the central monitoring system 130.

In one form, one or more of the first wireless communication system 112, the second wireless communication system 114, the third wireless communication system 122 and the fourth wireless communication system 116 can receive a plurality of signals from a plurality of local network nodes. For example, one or more of the first wireless communication system 112, the second wireless communication system 114, the third wireless communication system 122 and the fourth wireless communication system 116 can receive a first signal from a first network node, a second signal from a second network node and a third signal from a third network node. In the case where a signal is received from a plurality of network nodes, the controller 100 can estimate a radius at or within which the respiratory device may be located from each node. The controller 100 can then estimate the respiratory devices physical location relative to the plurality of network nodes by analyzing the data for each individual network node together. For example, the controller 100 can perform a trilateration calculation. The controller 100 can access the data from the memory indicative of the location of each network node from which a signal was received. The controller 100 can then estimate the physical location of the respiratory device. The controller 100 can estimate the relative location of the respiratory device with reference to the network nodes from which a signal was received, and, having information indicative of the physical location of each network node, the controller 100 can estimate the physical location of the respiratory device. When located on-board the respiratory device, the controller 100 can then transmit the estimated physical location of the respiratory device to the central monitoring system 130. The controller 100 can utilize one or more of the first, second, third and/or fourth wireless communication systems 112, 114, 122, 116 to transmit the location to the central monitoring system 130. In one form, the TDOA method may be performed using Long Range (LoRa) digital wireless data communication technology.

In another form, the respiratory device can collect the data and transmit said data to the central monitoring system 130 which comprises the controller 100, such that the TDOA method can be employed by the central monitoring system 130. Similar to as previously described, a signal sent from one or more nodes of a network, for example one or more WAPs 175 as previously described, cell towers or hardware modules 176 as previously described can include data comprising an identifier and a time. The identifier can uniquely identify the network node. The time can be the current time (e.g. the Coordinated Universal Time (UTC) time at which the signal was transmitted from each node). Alternatively, the time can be an internal system time that the respiratory device and nodes are synchronized to. The controller 100 can include a time keeping module as previously described.

One or more of the first, second, third and fourth wireless communication systems 112, 114, 122, 116 can receive the signal transmitted by each network node, and communicate the data of the signal to the controller 100. The controller 100 can then transmit via one or more of the first, second, third and fourth wireless communication systems 112, 114, 122, 116, some or all of the data received, and additional data to the central monitoring system 130 for processing. For example, the respiratory device can transmit the unique device identifier(s) received, the time at which the signal(s) was sent from the network node, and the time the signal(s) was received by the respiratory device (as detected at the respiratory device) to the central monitoring system 130. The controller 100 of the central monitoring system 130 can then perform at least one of the previously described locating calculations to estimate the location of the respiratory device.

In one form, the TDOA method may be performed using Long Term Evolution (LTE) Category M1 cellular technology. For example, the respiratory device may estimate the nearest cell tower (e.g. based on signal strength), and indicate the location of the respiratory device as the location of the cell tower.

In one form, the TDOA method may be performed using Narrowband Internet of Things (IoT) technology.

In one form, the location tracking method can be performed using Sigfox IoT technology.

In one form, the respiratory device can include a GPS module. The GPS module can comprise a GPS transceiver. The controller 100 can be configured to communicate with the GPS module to estimate the location of the respiratory device. The GPS module can be configured to communicate with one or more satellites. The GPS module can provide data indicative of the position of the satellites relative to the respiratory device, such that the controller 100 can use trilateration to estimate the location of the respiratory device.

In one form, the respiratory device can include a Random Phased Multiple Access (RPMA) module. For example, the respiratory device can include an Ingenu module. The RPMA module can operate in the 2.4 GHz frequency band.

In at least one form, the blower 6 can be removable from the respiratory device, and can comprise one or more of the wireless communication systems 112, 114, 122, 116. In such a configuration, the location of the blower 6 can be individually tracked according to one of the above methods.

In at least one form, the humidifier 12 can be removable from the respiratory device. The humidifier 112 can comprise one or more of the wireless communication systems 112, 114, 122, 116. In such a configuration, the location of the humidifier 12 can be individually tracked according to one of the above methods. For example, in a case where the humidification system 800 is used for respiratory therapy, the humidification system 800 can comprise one or more of the wireless communication systems 112, 114, 122, 116. In such a configuration, the location of the humidification system 800 may be independently tracked according to one of the above methods.

The previously described location tracking methods can be particularly useful to providers or users of respiratory devices. In some markets, a respiratory device provider can provide a plurality of respiratory devices to users on a temporary basis. For example, the respiratory device provider can provide loan respiratory devices to users to use at the user's own location (e.g. their home). The loan respiratory devices can be provided during a period of maintenance on the user's primary respiratory device, while the user is travelling, or for monetary compensation to reduce the up-front cost of respiratory device ownership to the user. The respiratory device provider has a large financial incentive to record and maintain data indicative of the location of each respiratory device that is provided on a temporary basis. Lost or stolen respiratory devices can reduce the profitability of the business model. The location tracking methods disclosed can significantly improve the ability of the respiratory devices provider to track and locate missing devices.

The previously described location tracking methods can also be particularly useful to providers of respiratory devices, even if the respiratory devices are used on the provider's premises. For example, hospitals can provide respiratory devices for use with patients of the hospital. Each patient can be provided their own respiratory device for the duration of their stay. The respiratory device can be moved with the patient throughout the hospital if they are moved. Following discharge of the patient, each respiratory device can be cleaned, and provided to another patient. If a respiratory device requires maintenance, a technician can transport the respiratory device to a maintenance area to perform the maintenance. Respiratory devices can be lost during patient movement, cleaning and/or maintenance. The location tracking methods disclosed can significantly improve the ability of the respiratory device provider (the hospital) to locate respiratory devices within the hospital's local network (e.g. the local Wi-Fi network). The location tracking system also prevents respiratory devices being lost or being recorded as lost, thereby reducing costs of locating and/or replacing lost respiratory devices.

The previously described location tracking methods can also be particularly useful to private users of respiratory devices. Private users can be individuals with respiratory conditions such as COPD who use their respiratory device at their home, or in a care environment such as a retirement facility or a palliative care facility. Alternatively, private users can be individuals with sleep conditions such as OSA who use their respiratory device at home while they sleep, or while they travel, for example in transit (e.g. on planes) or in temporary accommodation. In some cases, these private users can have multiple respiratory devices, one or more of which may not be used for extended periods of time. This can be the case if the user has a respiratory device specific to travel. The disclosed methods of location tracking allow the user to track the location of their respiratory device if they have misplaced it, or forgotten where it is after a period of non-use.

In one form, where the respiratory device comprises the blower 6, the controller 100 can turn off the blower 6 when the controller 100 estimates the respiratory device's location to be outside an expected operating perimeter. For example, the controller 100 can turn off the blower 6 when the controller 100 estimates the respiratory device is outside a boundary defined as the hospital grounds. Alternatively, the controller 100 can turn off the blower 6 when the controller 100 estimates the respiratory device is outside a boundary defined as a private user's property (e.g. a user's house).

In one form, where the respiratory device comprises the humidification system 800, the controller 100 can turn off the humidification system 800 when the controller 100 estimates the respiratory device's location to be outside an expected operating perimeter. For example, the controller 100 can turn off the humidification system 800 when the controller 100 estimates the respiratory device is outside a boundary defined as the hospital grounds. Alternatively, the controller 100 can turn off the humidification system 800 when the controller 100 estimates the respiratory device is outside a boundary defined as a private user's property (e.g. a user's house). Optionally the central monitoring system may be configured to provide an alarm message to another party associated with the respiratory device if the respiratory device is outside a predefined boundary. For example the central monitoring system may be configured to provide an alarm message to the owner of the respiratory device or a mobile device associated with the owner of the respiratory device if the respiratory device is detected be located outside a predefined boundary. In a further configuration the central monitoring system may be configured to provide a signal to disable the respiratory device if the respiratory device is detected outside a predefined boundary. Such a system can be useful to detect theft of the respiratory device.

Unless the context clearly requires otherwise, throughout the description and the claims, the words “comprise”, “comprising”, and the like, are to be construed in an inclusive sense as opposed to an exclusive or exhaustive sense, that is to say, in the sense of “including, but not limited to.”

The disclosed methods, apparatus and systems may also be said broadly to comprise the parts, elements and features referred to or indicated in the disclosure, individually or collectively, in any or all combinations of two or more of said parts, elements or features.

Although the present disclosure has been described in terms of certain embodiments, other embodiments apparent to those of ordinary skill in the art also are within the scope of this disclosure. Thus, various changes and modifications may be made without departing from the spirit and scope of the disclosure. For instance, various components may be repositioned as desired. Moreover, not all of the features, aspects and advantages are necessarily required to practice the present disclosure. Accordingly, the scope of the present disclosure is intended to be defined only by the claims that follow.

What is claimed is:

1. A medical device comprising:
a first power source, and
a location monitoring system for providing the location of the medical device, the location monitoring system comprising:
a dedicated location monitoring system comprising a second wireless communication system and a second power source configured to power said dedicated location monitoring system.

2. The medical device of claim 1, wherein the location monitoring system further comprises a primary system comprising a first wireless communication system.

3. The medical device of claim 1 or claim 2, wherein the first power source comprises a mains power connection and/or a first internal battery.

4. The medical device of claim 2, or claim 3 when dependent on claim 2, wherein the primary system is powered by a mains power connection.

5. The medical device of claim 3 or claim 4 when dependent on claim 3, wherein the primary system is powered by the first internal battery when the mains power connection is disconnected.

6. The medical device of any of claims 1-5, wherein the second power source is a second internal battery.

7. The medical device of any of claims 1-6, wherein the second wireless communication system comprises a global system for mobile communications, a cellular system, a Wi-Fi system, a global positioning system, or any combination thereof.

8. The medical device of claim 2 or any of claims 3-7, when dependent on claim 2, wherein the first wireless communication system comprises a global system for mobile communications, a cellular system, a Wi-Fi system, a global positioning system, or any combination thereof.

9. The medical device of any of claims 1-8, wherein the second wireless communication system comprises a global system for mobile communications and determines the location of said medical device using cell tower triangulation.

10. The medical device of any of claims 1-9, wherein the dedicated location monitoring system remains inactive for a period of time.

11. The medical device of claim 10, wherein the period of time is a predetermined period.

12. The medical device any of claims 1-11, further comprising a third wireless communication system, the third wireless communication system comprises a Wi-Fi system and determines the location of said medical device.

13. The medical device of any of claims 1-12, wherein the medical device is a respiratory device.

14. A method of monitoring a location of a medical device, the method comprising:
detecting a charge of a first internal battery is below a certain threshold;
determining the location of said medical device using a second wireless communication system; and
reporting the location of said medical device to a monitoring system at a predetermined frequency.

15. The method of claim 14, wherein the second wireless communication system comprises a global system for mobile communications, a cellular system, a Wi-Fi system, or a global positioning system, or any combination thereof.

16. The method of claim 14 or 15, wherein the frequency of reporting is reduced based on the charge of a second internal battery.

17. The method of any of claims 14-16, wherein the medical device is a respiratory device.

18. The method of any of claims 14-17, wherein a primary system is powered by a mains power connection.

19. The method of any of claims 14-17, wherein a primary system is powered by the first internal battery.

20. The medical device of any of claims 1-13, wherein the medical device is configured to perform the method of any of claims 14-19.

21. A method of monitoring a location of a medical device, the method comprising:
activating a dedicated location monitoring system after said dedicated location monitoring system remains inactive for a period of time;
determining a location of said medical device using the dedicated location monitoring system; and
reporting the location of said medical device to a central monitoring system.

22. The method of claim 20, the method further comprising determining the location of said medical device at a predetermined frequency.

23. The medical device of any of claims 1-13, wherein the medical device is configured to perform the method of claim 21, or claim 22 when dependent on claim 21.

24. A method of monitoring a location of a medical device, the method comprising:
defining a geographical boundary;
determining a location of said medical device using a wireless communication system;
detecting the medical device has left the geographical boundary; and
reporting the location of said medical device to a central monitoring system.

25. The medical device of any of claims 1-13, wherein the medical device is configured to perform the method of claim 24.

26. A method of monitoring a location of a medical device, the method comprising:
defining a geographical boundary;
determining a location of said medical device using a wireless communication system;
detecting the medical device has left the geographical boundary; and
disabling the medical device from further use.

27. The medical device of any of claims 1-13, wherein the medical device is configured to perform the method of any of claims 14-26.

28. A respiratory device configured to provide respiratory flow therapy to a user, the respiratory device comprising;
a housing comprising a gas inlet and a gas outlet,
a wireless communication system disposed within the housing, the wireless communication system configured to receive a first signal from a first wireless transmitter,
a controller configured to communicate with the wireless communication system, the controller being configured to receive the first signal and determine a first measure of a first parameter associated with the first wireless transmitter,
wherein the controller is configured to use the first measure to estimate a first location, the first location providing an indication of the location of the respiratory device relative to the first wireless transmitter,
wherein the controller is configured to retrieve a first transmitter physical location indicative of the physical location of the first wireless transmitter, and
wherein the controller is configured to estimate a device physical location using the first location and the first transmitter physical location.

29. The respiratory device of claim 28, wherein the controller is configured to communicate with a memory, and wherein the controller is configured to retrieve the first transmitter physical location from the memory.

30. The respiratory device of claim 28 or claim 29, wherein the memory is an on-board memory unit located within the housing.

31. The respiratory device of any one of claims 28-30, wherein the first parameter is associated with one or more of:
a) a received transmitter output as received by the wireless communication system, or
b) a time.

32. The respiratory device of claim 31, wherein the received transmitter output is a transmitter power output.

33. The respiratory device of claim 31 or claim 32, wherein the first measure is a measure of the received transmitter power output as received by the wireless communication system.

34. The respiratory device of any one of claims 28-33, wherein the controller is configured to compare the first measure to a first threshold, and the controller is configured to estimate the first location based on the comparison.

35. The respiratory device of claim 34, wherein the first threshold is a value indicative of the received transmitter power output as received by the wireless communication system at a predefined distance.

36. The respiratory device of any one of claims 33-35, wherein the controller is configured to apply a function to the first measure and the first threshold to estimate the first location.

37. The respiratory device of claim 31, wherein the first parameter is time, and the first measure is a transmission time indicative of the time the first signal was transmitted from the first wireless transmitter.

38. The respiratory device of claim 37, wherein the controller is configured to compare the transmission time to a received time, wherein the received time is indicative of a time the first signal was received by the wireless communication system, and the controller is configured to estimate the first location based on the comparison.

39. The respiratory device of claim 38, wherein the controller is configured to apply a function to the received time and the transmission time to estimate the first location.

40. The respiratory device of any one of claims 28-39, wherein the wireless communication system is configured to receive a signal from each of a plurality of wireless transmitters.

41. The respiratory device of claim 40, wherein the device receives a unique signal from each wireless transmitter.

42. The respiratory device of any one of claims 28-41, wherein the wireless communication system is configured to receive a second signal from a second wireless transmitter and a third signal from a third wireless transmitter.

43. The respiratory device of claim 42, wherein the controller is configured to determine, from the second signal, a second measure of a second parameter associated with the second wireless transmitter, and the controller is configured to determine, from the third signal, a third measure of a third parameter associated with the third wireless transmitter.

44. The respiratory device of claim 43, wherein each of the second parameter and the third parameter are associated with one or more of:
a) a received transmitter output as received by the wireless communication system, or
b) a time.

45. The respiratory device of claim 44, wherein the received transmitter output is a transmitter power output.

46. The respiratory device of claim 44 or claim 45, wherein the second measure is a measure of the received transmitter power output from the second wireless transmitter as received by the wireless communication system, and the third measure is a measure of the received transmitter power output from the third wireless transmitter as received by the wireless communication system.

47. The respiratory device of any one of claims 43-46, wherein the controller is configured to compare the second measure to a second threshold, and the controller is configured to estimate a second location based on the comparison, and wherein the controller is configured to compare the third measure to a third threshold, and the controller is configured to estimate a third location based on the comparison.

48. The respiratory device of claim 47, wherein the second threshold is a value indicative of the received transmitter power output from the second transmitter as received by the wireless communication system at a second predefined distance, and the third threshold is a value indicative of the received transmitter power output from the third transmitter as received by the wireless communication system at a third predefined distance.

49. The respiratory device of claim 47 or claim 48, wherein the controller is configured to apply a second function to the second measure and the second threshold to estimate the second location, and the controller is configured to apply a third function to the third measure and the third threshold to estimate the third location.

50. The respiratory device of claim 44, wherein the second parameter is time, and the second measure is a second transmission time indicative of the time the second signal was transmitted from the second wireless transmitter, and wherein the third parameter is time, and the third measure is a third transmission time indicative of the time the third signal was transmitted from the third wireless transmitter.

51. The respiratory device of claim 50, wherein the controller is configured to compare the second transmission time to a second received time, wherein the second received time is indicative of a time the second signal was received by the wireless communication system, and the controller is configured to estimate the second location based on the comparison.

52. The respiratory device of claim 50 or claim 51, wherein the controller is configured to compare the third transmission time to a third received time, wherein the third received time is indicative of a time the third signal was received by the wireless communication system, and the controller is configured to estimate the third location based on the comparison.

53. The respiratory device of claim 51, wherein the controller is configured to apply a second function to the second received time and the second transmission time to estimate the second location.

54. The respiratory device of claim 52, wherein the controller is configured to apply a third function to the third received time and the third transmission time to estimate the third location.

55. The respiratory device of any one of claims 43-54, wherein the controller is configured to use the first measure, the second measure and the third measure to estimate a relative location, the relative location providing an indication of the location of the respiratory device relative to the first wireless transmitter, the second wireless transmitter and the third wireless transmitter.

56. The respiratory device of any one of claims 43-54, wherein the controller is configured to perform a trilateration calculation to estimate a relative location, the relative location providing an indication of the location of the respiratory device relative to the first wireless transmitter, the second wireless transmitter and the third wireless transmitter.

57. The respiratory device of any one of claims 43-56, wherein the controller is configured to retrieve a first transmitter physical location indicative of a first physical location of the first wireless transmitter, a second transmitter physical location indicative of a second physical location of the second wireless transmitter, and a third transmitter physical location indicative of a third physical location of the third wireless transmitter.

58. The respiratory device of claim 57, wherein the controller is configured to estimate the device physical location within a localized environment using the relative location, the first transmitter physical location, the second location, the second transmitter physical location, the third location and the third transmitter physical location.

59. The respiratory therapy device of any one of claims 28-58, wherein the controller is configured to estimate the device physical location using trilateration.

60. The respiratory device of any one of claims 28-59, wherein the controller is configured to communicate with a remote memory via the wireless communication system.

61. The respiratory device of any one of claims 28-60, wherein the wireless communication system comprises one or more of:
a) a Wi-Fi system,
b) a cellular network system,
c) a GSM system, or
d) a BlueTooth® system.

62. The respiratory device of any one of claims 28-61, wherein the respiratory device comprises a blower disposed within the housing, the blower configured to deliver air from the gas inlet to the gas outlet.

63. The respiratory device of any one of claims 28-62, wherein the respiratory device comprises a humidification system.

64. The respiratory device of any one of claims 28-63, wherein the controller is configured to turn off the blower when the controller estimates the respiratory device's location to be outside an expected operating perimeter.

65. The respiratory device of any one of claims 28-64, wherein the controller is configured to turn off the humidification system when the controller estimates the respiratory device's location to be outside an expected operating perimeter.

66. The respiratory device of any one of claims 28-65, wherein the controller is configured to transmit an alarm using the wireless communication system to a central monitoring system indicating the estimated location of the respiratory device.

67. The respiratory device of any one of claims 28-66, wherein the respiratory therapy device is configured to output an audible alarm when the controller estimates the respiratory device's location to be outside an expected operating perimeter.

68. The respiratory device of any one of claims 28-67, wherein the respiratory device is one or more of:
a) a continuous positive airway pressure device,
b) a Bi-Level positive airway pressure device,
c) a nasal high-flow device,
d) a non-invasive ventilation device, and
e) a ventilator.
